(19) United States
(12) Patent Application Publication
(10) Pub. No.: US 2010/0016364 A1
(43) Pub. Date: Jan. 21, 2010

(54) METHOD OF PREDICTIVE DETERMINATION OF RESPONSIVENESS TO PHARMACOLOGICAL INTERVENTION

(76) Inventors: Roger K. Cady, Ozark, MO (US); Paul L. Durham, Springfield, MO (US)

Correspondence Address: HUSCH BLACKWELL SANDERS LLP, 190 Carondelet Plaza, Suite 600, ST. LOUIS, MO, 63105, US

(21) Appl. No.: 12/556,677

(22) Filed: Sep. 10, 2009

Related U.S. Application Data
(63) Continuation-in-part of application No. 11/863,369, filed on Sep. 28, 2007.
(60) Provisional application No. 60/827,340, filed on Sep. 28, 2006.

(51) Int. Cl. A61K 31/454; C12Q 1/68; G01N 33/53; A61K 31/421; A61K 31/4196; A61K 31/403; A61K 31/4045; C07K 14/00

(52) U.S. Cl. 514/323; 435/6; 435/7.1; 514/376; 514/383; 514/411; 514/414; 514/415; 530/350

(57) ABSTRACT

A diagnostic test for determining the efficacy of 5HT₁ agonists in a human patient exhibiting symptoms of a cranial facial pain syndrome includes the steps of collecting a sample of saliva from said human patient; and identifying whether calcitonin-gene-related peptide is present in said sample of saliva.

### CROSS REFERENCE

This application claims the priority of co-pending utility application Ser. No. 11/863,369 filed Sep. 28, 2007, which in turn claims the priority of provisional application Ser. No. 60/827,340, filed Sep. 28, 2006.

### BACKGROUND OF THE INVENTION

The present invention relates generally to pharmacology and more particularly to a method of determining the efficacy of certain cranial facial pain syndromes medications in individual human patients and a related method of treating migraine.

### BACKGROUND OF THE INVENTION

Facial pain disorders are common and cause substantial disability and work absenteeism. For example, migraine affects an estimated 12% of the adult population and is estimated to account for over 150 million days of work absenteeism. Beyond migraine, other common head and facial pain disorders include tension headache, migrainous headache, cluster headache, and sinusitis and tempromandibular joint dysfunction. Also, asthma and rhinosinusitis are other common and disabling medical conditions.

The differentiation of these disorders is largely based on clinical symptomatology and as such is a common cause of misdiagnosis which leads to inappropriate treatment. In addition there is a medical need to gauge the severity and progression of these diseases over time.

Furthermore, while 5HT₁ agonists or triptans have been previously disclosed as an effective means for acute treatment and in some instances prophylaxis of migraine, as disclosed, for example, in U.S. Pat. No. 7,189,753, the disclosure of which is incorporated in its entirety by reference herein, the effectiveness of triptans has been found to vary from individual to individual and even from attack to attack for a given individual. Therefore, there is a need to identify a means for determining on a patient-by-patient basis whether triptans, in particular, but also other known migraine medications, will be an effective treatment option for a given patient.

The present invention is directed to overcoming one or more of the problems set forth above.

### SUMMARY OF THE INVENTION

One aspect of the invention generally pertains to a method for a rapid and non-invasive diagnostic testing method for headache and facial pain that is predictive of the effectiveness of certain known medications, in particular, triptans and other medications known to have a mechanism of action that involves calcitonin gene-related peptide.

Another aspect of the invention is to provide a method for rapid and non-invasive diagnostic testing for specific biological markers relevant to the diagnosis and monitoring of headache and facial pain.

Yet another aspect of the invention is to provide an improved method for treating incidents of migraine in individual human patients.

In one embodiment of the invention, there is provided a diagnostic test for determining the efficacy of 5HT₁ agonists in a human patient exhibiting symptoms of a cranial facial pain syndrome that includes the steps of collecting a sample of saliva from said human patient and identifying whether calcitonin-gene-related peptide is present in said sample of saliva.

In another embodiment, there is provided a method for classifying whether a human patient exhibiting symptoms of a cranial facial pain syndrome is likely to respond to administration of a 5HT₁ agonist that includes obtaining a biological sample from the patient; and determining a diagnostic marker profile by detecting the presence or level of calcitonin-gene-related peptide in the biological sample.

In another embodiment, there is provided a biological marker for determining responsiveness of a human patient exhibiting symptoms of a cranial facial pain syndrome to administration of a 5HT₁ agonist that comprises calcitonin-gene-related peptide.

In another embodiment, there is provided a method for determining the efficacy of 5HT₁ agonists for treatment of a cranial facial pain syndrome that includes the steps of evaluating a biological sample obtained from a subject exhibiting symptoms of the cranial facial pain syndrome for the presence or absence of calcitonin-gene-related peptide, wherein the presence of calcitonin-gene-related peptide in the sample indicates that the subject is likely to respond to administration of 5HT₁ agonists.

In another related embodiment, there is provided a preemptive prophylaxis method for treatment of a cranial facial pain syndrome in a human patient that includes the steps of collecting a sample of saliva from the patient; evaluating the saliva sample for evidence of the presence of calcitonin-gene-related peptide to determine an efficacy of a 5HT₁ agonist in treating the cranial facial pain syndrome in the patient; and administering the 5HT₁ agonist to the patient if the evaluation of the saliva sample indicates that such treatment will be effective.

These aspects are merely illustrative of the innumerable aspects associated with the present invention and should not be deemed as limiting in any manner. These and other aspects, features and advantages of the present invention will become apparent from the following detailed description when taken in conjunction with the referenced drawings.

### BRIEF DESCRIPTION OF THE DRAWINGS

Reference is now made more particularly to the drawings, which illustrate the best presently known mode of carrying out the invention and wherein similar reference characters indicate the same parts throughout the views.

FIG. 1 is a schematic diagram of a diagnostic test for head and facial pain according to one embodiment of the present invention.

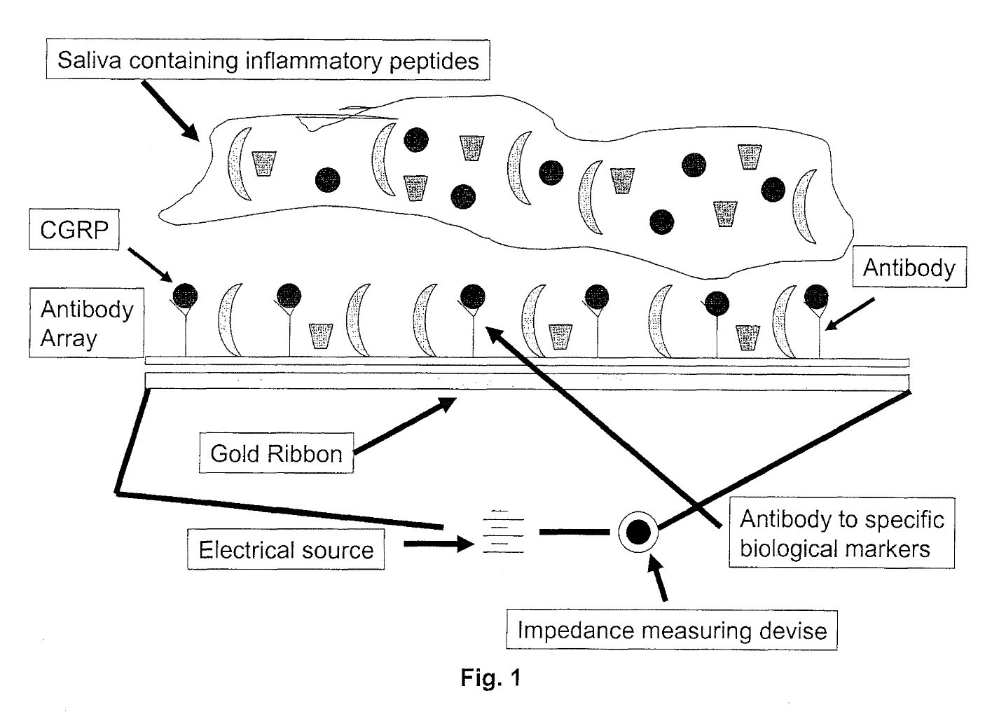

FIG. 2 is a graph illustrating levels of CGRP in human saliva collected from all study participants during various phases of migraine as well as non-headache and post-migraine resolution periods.

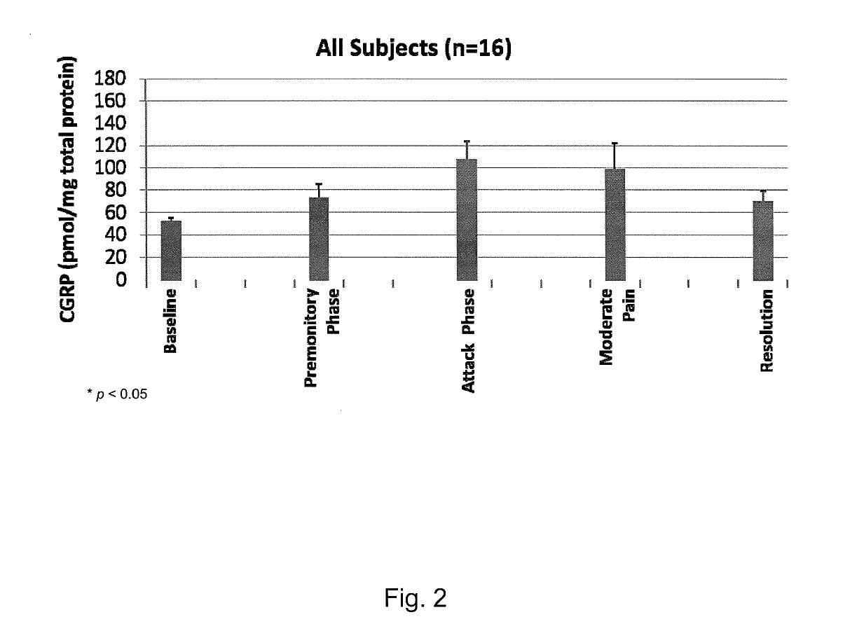

FIG. 3 is a graph illustrating levels of CGRP among study participants who responded to triptan treatment during various phases of migraine as well as non-headache and post-migraine resolution periods.

FIG. 4 is a graph illustrating levels of CGRP among study participants who did not respond to triptan treatment during various phases of migraine as well as non-headache and post-migraine resolution periods.

FIG. 5 is a graph illustrating the change relative to baseline measurements of all study participants during various phases of migraine.

FIG. 6 is a graph illustrating the change relative to baseline measurements of study participants who responded to triptan treatment during various phases of migraine.

FIG. 7 is a graph illustrating the change relative to baseline measurements of study participants who responded to triptan treatment and experienced an increase of CGRP levels at the attack onset phase of migraine.

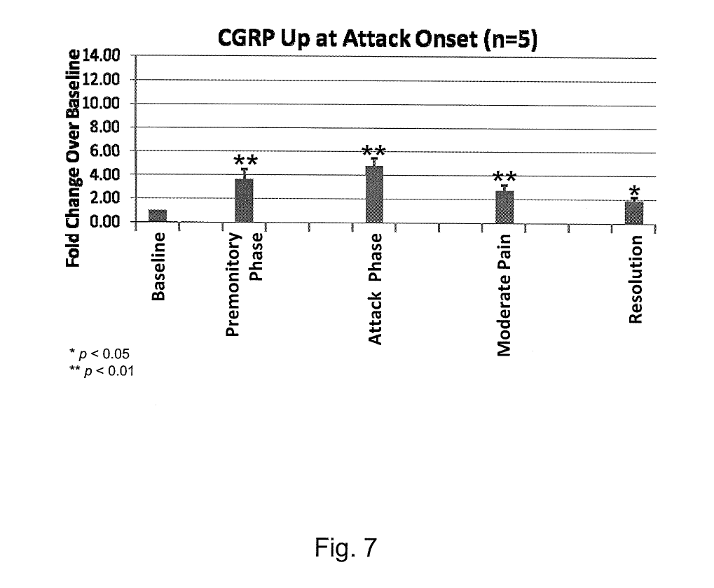

FIG. 8 is a graph illustrating the change relative to baseline measurements of study participants who responded to triptan treatment and experienced an increase of CGRP levels at the moderate pain phase of migraine.

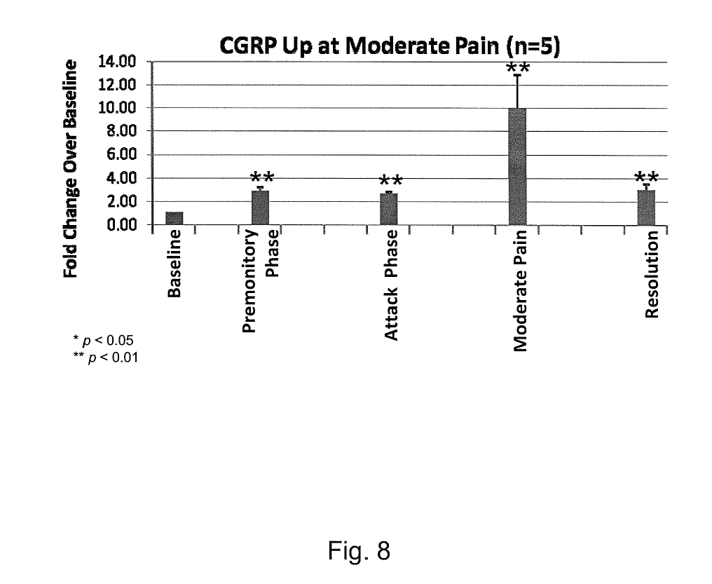

### DESCRIPTION OF THE PREFERRED EMBODIMENTS

In the following detailed description numerous specific details are set forth in order to provide a thorough understanding of the invention. However, it will be understood by those skilled in the art that the present invention may be practiced without these specific details. For example, the invention is not limited in scope to the particular type of industry application depicted in the figures. In other instances, well-known methods, procedures, and components have not been described in detail so as not to obscure the present invention.

Inflammatory peptides, proteins, human growth factors, endorphins, enkephalines, prostaglandins, and a variety of cytokines and other biological markers released during migraine, sinus headache, and rhinosinusitis can be measured in the salvia of an affected patient in a specific pattern that permits identification of the underlying pathophysiology. In addition, changes in inflammatory peptide patterns between attacks of episodic disease are uniquely different from those observed in patients with chronic disease patterns. Furthermore, saliva can be used as a diagnostic substrate containing biological markers for measuring these changes.

The method described herein can identify, differentiate, diagnose, and stage the progression of disease for the purpose of directing appropriate treatment of diseases and disorders of headache and facial pain that involve the release of biological markers such as calcitonin-gene-related peptide (“CGRP”), vasoactive intestinal peptide, neurokinin A and B, substance P, c-reactive protein, amylase, IgG, IgA, nitric oxide, prostaglandins, and histamine as a component of the disease process. The testing method can be applied to respiratory diseases, such as asthma and rhino sinusitis. In particular, recent preliminary clinical studies have been conducted with respect to the correlation of CGRP levels in biological samples of saliva collected from patients with a history of or exhibiting symptoms of migraine. The results of these studies clearly suggest that such a correlation in fact exists.

Research has shown 5-HT₁ receptor agonists, sometimes referenced as triptans, to be useful in the treatment of migraine. 5-HT₁ receptors are located, for example, in the dog saphenous vein and the 5HT₁ receptor agonists with which the present invention is concerned contract the dog saphenous vein. Such compounds may therefore be identified by their contractile effect on the dog isolated saphenous vein strip as described, for example, by Apperley et al, Br. J. Pharmacol, 68, 215-224 (1980). Compounds which are selective 5HT₁ receptor agonists have also been found to selectively constrict the carotid arterial bed of the anaesthetized dog. 5HT₁ receptors are also found in human trigeminal nerves and throughout the central nervous system.

Much work has been done in attempts to identify the subclasses of 5HT₁ receptors which are implicated in migraine. It is currently thought that 5HT_{1B} (formerly 5HT_{1Dβ}), 5HT_{1D} (formerly 5HT_{1Dα}) and 5HT_{1F} receptors are particularly important. Tests in isolated cerebral arteries can be used to determine which of these receptor sub-types mediate the action of 5HT₁ agonist compounds, for example as described in Bouchelet, I. et al, Mol. Pharmacol 1996, 50, 219-223.

Some 5HT₁ agonists, including 5HT_{1D} and 5HT_{1F} agonists have also been found to inhibit the trigeminal nerve. This can be assessed by measuring plasma protein extravasation in the dura mater following trigeminal nerve stimulation and administration of labeled albumin; active compounds produce inhibition of dural plasma protein extravasation in this model, which is described in Buzzi. M. G and Moskowitz M. A, Br. J. Pharmacol, 1990 99, 202-206.

A variety of compounds have been identified in the art as 5HT₁ agonists, for example by selective constriction of the dog isolated saphenous vein or constriction of the carotid arterial bed of the anaesthetized dog. These include indole derivatives such as those disclosed inter alia in: published British Patent Specification Nos. 2082175, 2081717, 2083463, 2124210, 2150932, 2162522, 2168347, 2168973, 2185020, 2186874, 2191488, 2008646, 2289464, 2289465, 2286185; published U.S. Pat. Nos. 5,288,748, 5,317,103, 5,382,592, 5,385,928, 5,387,593, 5,418,236, 5,433,915, 5,451,584, 5,466,688, 5,468,768, 5,519,025, 5,545,644, 5,602,128, 5,618,948, 5,637,611, 5,837,715; published German Patent Specification Nos. 4414113, 4226527; published French Patent Specification Nos. 2699918, 2707294, 2737723; published Japanese Patent Specification Nos. 05320157, 06041071, 07097370, 07109265; published European Patent Specification Nos. 147107, 237678, 242939, 244085, 225726, 254433, 303506, 313397, 354777, 382570, 464558, 506363, 506369, 450238, 451022, 451008, 478954, 438230, 494774, 497512, 501568, 580539, 581538, 586866, 590970, 590971, 603432, 610134, 620222, 620223, 644187, 641787, 645385, 648767, 666258, 668273, 683155, 700905, 703229, 707007, 708102, 712837, 714894, 714896, 729958, 733628, 736525, 747353, 749962, 755932, 768301, 810220; and published International Patent Application Nos. WO92/11013, WO92/11014, WO92/06973, WO93/00086, WO92/13856, WO93/00094, WO91/18897, WO93/00333, WO94/02460, WO94/02477, WO94/03446, WO94/06789, WO94/10171, WO94/11363, WO94/11356, WO94/13620, WO94/14773, WO94/14772, WO94/14771, WO94/14770, WO94/15930, WO94/14779, WO94/18193, WO94/21619, WO94121611, WO94/21610, WO94/20466, WO94/24127, WO94/29293, WO95/01334, WO95/01965, WO95/05383, WO95/05381, WO95/05366, WO95/06636, WO95/11903, WO95/20588, WO95121167, WO95/21166, WO95/28933, WO95/32196, WO96/04274, WO96/04269, WO96/06846, WO96/06638, WO96/09288, WO96/11195, WO96/11930, WO96/11923, WO96/11685, WO96/12721, WO96/12713, WO96/16056, WO96/17842, WO96/17831, WO96/16961, WO96/16949, WO96/23789, WO96/23784, WO96/23785, WO96/24596, WO96/29075, WO96/39133, WO97/41802, WO97/03068, WO97/08159, WO97/11695, WO97/13512, WO97/11946, WO97/11945, WO97/17343, WO97/17338, WO97/17337, WO97/18204, WO97/18203, WO97/18202, WO97/18201, WO97/16446, WO97/19073, WO97/38692, WO97/43281, WO97/42189, WO97/45426, WO95/14004, WO98/12183, WO98/14433, WO98/15545. The compounds disclosed in the aforementioned specifications (hereinafter described as “Y compounds”) are described as being useful in the treatment of migraine and cluster headache. Some are also claimed to be useful in treating tension-type headache.

Specific examples of 5HT₁ agonist Y compounds include those indole derivatives often called “triptans” such as the compounds with the generic names: sumatriptan, naratriptan, rizatriptan, zolmitriptan, eletriptan, almotriptan and frovatriptan. Other examples of 5HT₁ agonist Y compounds include those commonly known as ALX-0646, LY334370, U109291, 1S159 and PNU-142633.

While triptans have been found to be useful in the treatment of migraine, as disclosed in U.S. Pat. No. 7,189,753, their effectiveness at the clinical level has been found to vary from individual to individual. In fact, anecdotal evidence suggests that as much as one-third (⅓) of all migraine patients fail to respond to triptans. One aspect of the present invention provides a useful test for determining the efficacy of triptans in treating migraine in individual patients. In particular, it has been found that those patients in which the biological marker calcitonin-gene-related peptide (“CGRP”) is found in the saliva of the patient are responsive to pharmacological intervention with triptans. Thus, the previously described diagnostic method advantageously presents a means for detecting the presence of CGRP and, therefore, determining the efficacy of intervention with triptans.

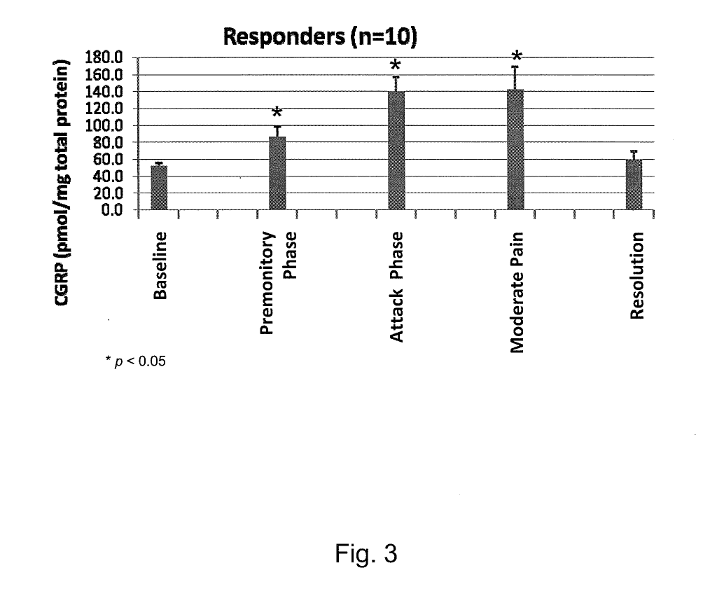

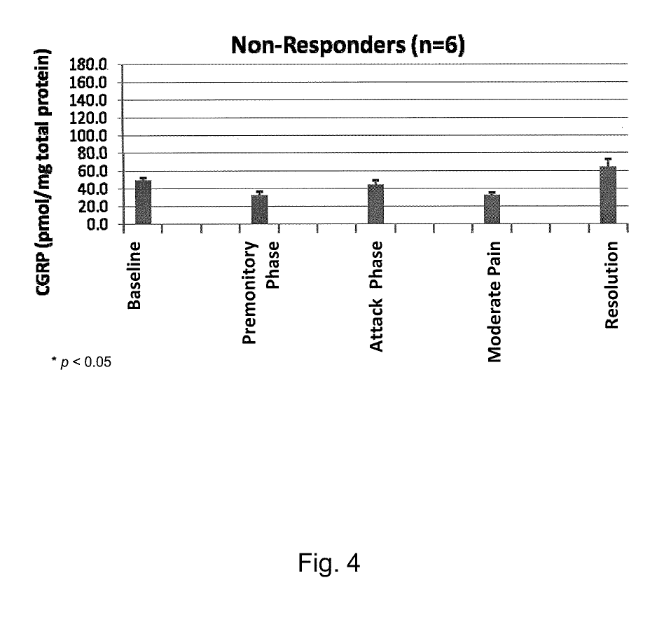

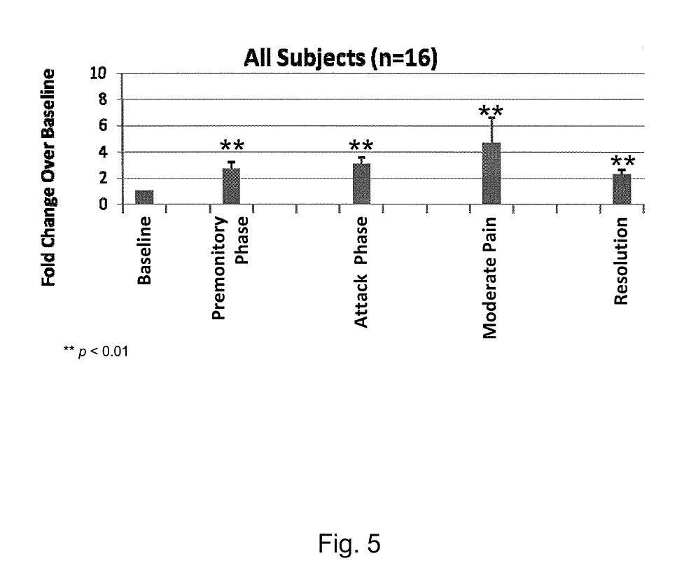

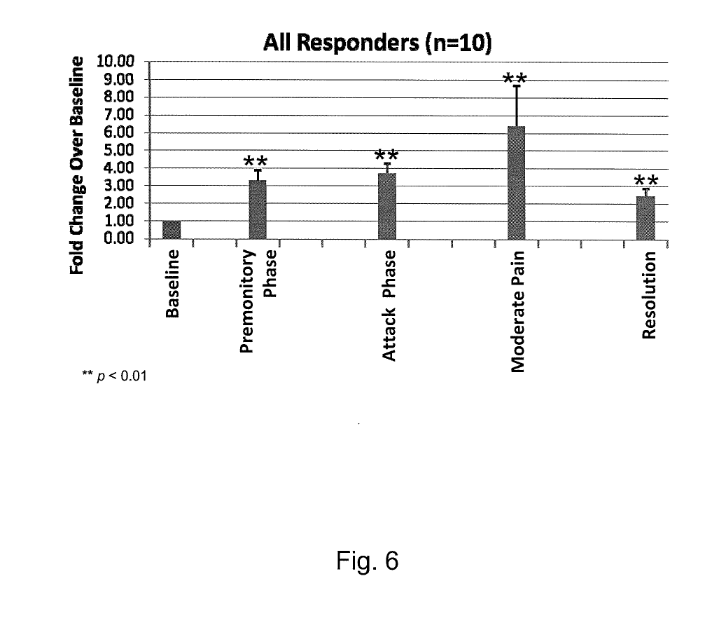

The clinical studies referenced above provide support for this theory. In these studies, following collection and analysis of saliva samples, the participants were administered identical doses of triptans and participants who responded to such triptan treatment were identified. When analyzing the data from these studies, the saliva CGRP levels of participants who responded to triptan treatment (“responders”) were compared to the corresponding levels in participants who did not respond to the administered triptan treatment (“non-responders”). The results of this analysis are illustrated graphically in FIGS. 3 and 4. While the CGRP levels in the saliva of both groups were generally similar in baseline measurements taken before onset of any symptoms of migraine and in measurements taken following complete resolution of the migraine episode, a significant distinction can be seen during the various phases of a migraine episode. During migraine episodes, the CGRP levels of responders were generally between 2 to 4 times the levels observed in non-responders. As shown in FIGS. 5 and 6, the degree of change in CGRP levels between a baseline, non-migraine measurement and during migraine episodes increases dramatically for responders (FIG. 6) relative to the degree of change exhibited by the study participants as a whole (FIG. 5).

Based on this information, it is believed that the methodology disclosed herein provides the opportunity to identify in advance the potential efficacy of triptans and other 5HT₁ agonists on a patient by patient basis. Furthermore, due to the possibility for variability in the effectiveness of triptan-based treatment from migraine to migraine for a given individual, the relative simplicity of the testing methodology is well-suited for home-based as well as office or laboratory centered testing regimes.

This methodology is effective both from a positive perspective, i.e., identifying those patients for whom triptans will constitute an effective migraine treatment, and a negative one. From a negative aspect, the absence of CGRP or presence of only limited levels of CGRP in a sample will indicate that a different migraine mechanism is in play in the corresponding patient and that alternative therapies should be considered.

The primary goal of the referenced studies was to compare baseline levels of CGRP in saliva samples of study participants from migraine free periods with CGRP levels observed during (a) the premonitory phase of migraine, (b) the mild headache phase (within two (2) hours of onset), (c) moderate to severe headache, and (d) post-treatment (within four (4) hours of migraine resolution). Secondary goals of the study included:
Measure and compare changes in CGRP levels with occurrence of non-headache migraine symptomatology, such as, nausea, photophobia, phonophobia, and muscle pain
Measure and compare changes in CGRP as a function of premonitory symptoms
Measure and compare changes in CGRP as a function of the subjects' ability to predict a moderate to severe attack of migraine from early symptoms of migraine
Compare premonitory symptoms recorded at screening to those recorded at the time of treatment
Measure and compare levels of CGRP obtained in the premonitory or mild headache phase that did not progress to moderate to severe migraine with CGRP levels obtained in the premonitory and mild headache phase of migraine attacks that did progress to moderate to severe headache

The study focused upon healthy subjects with episodic migraine (IHS 1.1 or 1.2) who had not utilized preventive medications in the previous three months. Subjects had a history of at least 15 days per month without primary headache for the previous three months; were aware of premonitory symptoms with at least 75% of migraine attacks; experienced a mild headache phase with a least 75% of their migraine attacks; and were willing to treat an attack of migraine when the headache is moderate to severe in intensity. Subjects also had a history of 1-6 migraines per month within the past 3 months and at least 15 headache free days throughout the previous three month period. Subjects were required to keep diary records to record time of symptom assessment, symptoms being experienced, and subject's assessment as to the likelihood that a current migraine would progress to moderate or severe headache.

Baseline levels of CGRP were obtained during a headache free time (no headache within the previous 48 hours). All subjects completed a detailed questionnaire regarding premonitory and early headache symptomatology. Saliva collection occurred at five (5) time points: (1) during screening when the subject had been free of primary headaches for at least 48 hours; (2) early in the premonitory phase once the subject predicted moderate to severe headache; (3) within 2 hours of the onset of mild headache; (4) before treatment of moderate to severe headache with a triptan; (5) within 4 hours of migraine resolution. At each collection of saliva, the subject recorded symptoms. During the premonitory and mild headache phase, the subject rated the probability that the current migraine would become associated with a moderate to severe headache. Saliva samples obtained for symptoms suggesting early migraine but not becoming severe enough to require treatment were compared with CGRP levels in attacks that did evolve to moderate to severe headache.

The investigator then instructed each subject to treat the next attack of migraine with a triptan within one (1) hour of onset of moderate to severe headache. Migraine attacks awakening the subject were excluded from analysis. Rescue at 2 hours post treatment was permitted with medication determined by the investigator.

Saliva collection was conducted both in clinic during screening and at the participant's home during treatment. The participants were directed to avoid chewing gum, consumption by mouth of any substance other than water, and smoking for the two hours preceding saliva collection. Participants were also instructed to thoroughly rinse their mouths prior to sample collection. Saliva was collected after stimulation of the tip and sides of the tongue with 2% citric acid applied via a cotton swab every 30 seconds for several minutes. Collected saliva was primarily generated from the submandibular and sublingual glands. When necessary, Stenson's and Wharton's ducts were blocked with gauze to limit collection from those sources. Stimulated saliva was preferred due to the fact that sufficient, more consistent quantities can be collected in a shorter period of time. Furthermore, control of unstimulated saliva is more difficult as a wide range of mechanical or sensory stimuli can active salivary gland secretion to levels above the normal basal state. Saliva produced during the first two minutes following initiation of stimulation was discarded to minimize the intermingling of stimulated and unstimulated saliva.

Collected saliva samples were immediately placed in a freezer and stored for analysis. In the case of at home sample collection, participants were instructed to return samples to the clinic within 72 hours of collection.

Collected saliva samples were centrifuged at 3,000 rpm at 4° C. for thirty (30) minutes. The resultant supernatant was aliquoted into 1.5 mL centrifuge tubes and stored at −20° C. Prior to protein analysis, samples were thawed slowly and then centrifuged for 3 minutes at 13,200 rpm at room temperature to pellet cellular debris. The total amount of protein in each sample was determined using the Bradford assay. CGRP levels in the samples were determined through peptide-specific radioimmunoassay. The immunoassay kits utilized in the study were acquired from Bachem Peninsula Labs. The total peptide content (pmol) for each sample was calculated by dividing the amount of total peptide by the molecular weight of the specific peptide, CGRP (3789 g/mol). Finally, reported peptide values were normalized by adjusting for total volume (mL) and protein (mg) in each sample.

Each experimental condition was assayed in duplicate and a statistical analysis performed. Values of CGRP were grouped according to the corresponding phase of migraine in which the collection occurred: baseline, premonitory phase, mild headache phase, moderate to severe headache phase, and post treatment. Mean values and standard deviations were then determined. A Kruskal-Wallis one-way ANOVA compared the CGRP levels obtained for the different groups. CGRP levels were plotted and analyzed using the variable of time of onset of first awareness (premonitory symptoms) of migraine to time of treatment with a triptan. As an exploratory endpoint, CGRP levels were compared for migraine not evolving to a moderate to severe headache and those that did evolve to a moderate to severe headache.

The described methodology is advantageously well-suited to a known biosensor that has been developed. Application of the present method to such a biosensor permits measurement of changes in several biological markers in human saliva that occur with specific disease processes.

The method consists of providing a series of antibodies to several biological markers on a test strip in a known manner, including inflammatory peptides, proteins, human growth factors, endorphins, enkephalines, prostaglandins, and a variety of cytokines. A sample of saliva is collected from a human to be tested and applied to the series of antibodies. If the anticipated biological markers are present in the sample of saliva, those markers bind to their correlating antibodies thereby providing an indicator of the presence and amount of a particular biological marker in the patient's saliva upon evaluation of the test strip. A baseline sample may be taken and compared to subsequent samples in order to determine changes in the levels of specific biological markers in the patient over time.

When applied to a suitable biosensor, the described method can generate an electrical signal that can subsequently be transmitted to other devices. The biosensor consists of a substrate that is coated with D-poly lysine on which antibodies to various biological markers are randomly arranged. In preferred embodiment, the substrate comprises a gold ribbon. When saliva is applied to the biosensor, a biological marker present in the saliva will bind to its correlating antibody. The interaction of biological marker and antibody alters the electrical impedance of the substrate. This change in impedance can be readily measured and converted into a transmittable electrical signal. It is possible to use various salivatory proteins with this methodology to provide measurement and identification of biological markers critical in diagnosis, staging of disease progression, and treatment of many head and facial pain disorders.

In addition to the biosensor and the antibody evaluation methodology utilized in the clinical studies described herein, various forms of liquid chromatography-mass spectrometry that are well known to those of skill in the art re suitable for use with the disclosed method. In particular, capillary liquid chromatography-electrospray ionization-mass spectrometry and tandem mass spectrometry has been utilized successfully. High performance liquid chromatography mass spectrometry is an extremely versatile instrumental technique whose roots lie in the application of more traditional liquid chromatography to theories and instrumentation that were originally developed for gas chromatography (GC). The required instrumentation includes a high performance liquid chromatograph (HPLC) attached, via a suitable interface, to a mass spectrometer (MS). Compounds that are thermally labile, exhibit high polarity or have a high molecular mass, including proteins, may all be analyzed using HPLC/MS. Solutions derived from samples of interest are injected onto an HPLC column that comprises a narrow stainless steel tube (usually 150 mm length and 2 mm internal diameter, or smaller) packed with fine, chemically modified silica particles. Compounds are separated on the basis of their relative interaction with the chemical coating of these particles (stationary phase) and the solvent eluting through the column (mobile phase). Components eluting from the chromatographic column are then introduced to the mass spectrometer via a specialized interface. The most common interface used for HPLC/MS is electrospray ionization.

In electrospray ionization the analyte is introduced to the source at flow rates typically of the order of 1 μl min⁻¹. The analyte solution flow passes through an electrospray needle that has a high potential difference (with respect to the counter electrode) applied to it (typically in the range from 2.5 to 4 kV). This forces the spraying of charged droplets from the needle with a surface charge of the same polarity to the charge on the needle. The droplets are repelled from the needle towards the source sampling cone on the counter electrode. As the droplets traverse the space between the needle tip and the cone, solvent evaporation occurs. As the solvent evaporation occurs, the droplet shrinks until it reaches the point that the surface tension can no longer sustain the charge (the Rayleigh limit) at which point a “Coulombic explosion” occurs and the droplet is ripped apart. This produces smaller droplets that can repeat the process as well as naked charged analyte molecules. These charged analyte molecules can be singly or multiply charged. This is a very soft method of ionization as very little residual energy is retained by the analyte upon ionization. It is the generation of multiply charged molecules that enables high molecular weight components such as proteins to be analyzed since the mass range of the mass spectrometer is greatly increased since it actually measures the mass to charge ratio rather than mass per se. The major disadvantage of the technique is that very little (usually no) fragmentation is produced although this may be overcome through the use of tandem mass spectrometric techniques such as MS/MS or MSⁿ.

In addition to the previously described evaluation methodologies, embodiments of the present invention may also utilize what is known as “spiegelmer” technology. Speigelmers are sometimes referenced as “mirror imaged aptamers” and are indeed a direct derivative of aptamers. Aptamers are oligonucleotide ligands that can be generated to bind to targets with high affinity and specificity. Aptamers are based on the Systematic Evolution of Ligands by Exponential enrichment process (“SELEX”). They are isolated by in vitro selection from combinatorial nucleic acid libraries that are composed of a central randomized region and additional fixed primer binding sites with ˜30-40 nt. The fixed primer binding sites permit amplification by polymerase chain reactions (PCR). Aptamers have binding characteristics similar to peptides or antibodies, with affinities in the low nanomolar to the picomolar range. One disadvantage of aptamers is that they are prone to rapid degradation by nucleases that are present in all human tissues.

Spiegelmer technology was developed in order to address this deficiency of aptamers. Spiegelmers are biostable aptamers and have similar diversity characteristics while proving resistant to enzymatic degradation. In particular, aptamers are created from D-nucleotides, while spiegelmers are synthesized from mirror image L-oligonucleotides. There are no known enzymes in human tissues that are capable of interacting with L-oligonucleotides. Spiegelmer technology was developed by NOXXON Pharma AG and is the subject of several issued patents and pending applications. In particular, U.S. Publication No. US2006/0183700 describes the use of spiegelmer technology in the creation of antagonists for CGRP. The disclosure of U.S. Publication No. US2006/0183700 is expressly incorporated by reference herein in its entirety.

Spiegelmer technology is utilized within the general concept of embodiments of the current invention by being used to create a nucleic acid based binding agent for CGRP to be utilized in evaluating biological samples for the presence of CGRP. Spiegelmers have the benefit of being stable in human biological samples for extended periods of time, which facilitates the evaluation process described herein. While Spiegelmers have here to date been used almost exclusively in drug discovery platforms, the presently described embodiment of the invention is believed to be the first use of this technology for a diagnostic purpose.

The preferred embodiments of the invention have been described above to explain the principles of the invention and its practical application to thereby enable others skilled in the art to utilize the invention in the best mode known to the inventors. However, as various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by the above-described exemplary embodiment, but should be defined only in accordance with the following claims appended hereto and their equivalents.

I claim:

1. A diagnostic test for determining the efficacy of 5HT₁ agonists in a human patient exhibiting symptoms of a cranial facial pain syndrome, comprising the steps of:
collecting a sample of saliva from said human patient; and
identifying whether calcitonin-gene-related peptide is present in said sample of saliva.

2. The diagnostic test for determining the efficacy of 5HT₁ agonists as set forth in claim 1 further comprising the step of stimulating production of saliva in said human patient prior to collecting said sample of saliva.

3. The diagnostic test for determining the efficacy of 5HT₁ agonists as set forth in claim 2, wherein said step of stimulating production of saliva further comprises stimulating production of saliva from the submandibular and sublingual glands of said human patient.

4. The diagnostic test for determining the efficacy of 5HT₁ agonists as set forth in claim 1, wherein the step of identifying whether calcitonin-gene-related peptide is present further comprises evaluating said sample of saliva by radioimmunoassay.

5. The diagnostic test for determining the efficacy of 5HT₁ agonists as set forth in claim 1, wherein the step of identifying whether calcitonin-gene-related peptide is present further comprises evaluating said sample of saliva by liquid chromatography-mass spectrometry.

6. The diagnostic test for determining the efficacy of 5HT₁ agonists as set forth in claim 1, wherein the step of identifying whether calcitonin-gene-related peptide is present further comprises providing a nucleic acid corresponding to calcitonin-gene-related peptide and evaluating said saliva sample for evidence of binding of said nucleic acid with calcitonin-gene-related peptide present in said saliva sample.

7. The diagnostic test for determining the efficacy of 5HT₁ agonists as set forth in claim 1, wherein said nucleic acid is a mirror-imaged aptamer.

8. The diagnostic test for determining the efficacy of 5HT₁ agonists as set forth in claim 1, wherein the step of identifying whether calcitonin-gene-related peptide is present further comprises providing an antibody corresponding to calcitonin-gene-related peptide and evaluating said saliva sample for evidence of binding of said antibody with calcitonin-gene-related peptide present in said saliva sample.

9. The diagnostic test for determining the efficacy of 5HT₁ agonists as set forth in claim 1, wherein the presence of calcitonin-gene-related peptide in said biological sample indicates that the subject is likely to respond to administration of 5HT₁ agonists.

10. The diagnostic test for determining the efficacy of 5HT₁ agonists as set forth in claim 1, wherein the absence of calcitonin-gene-related peptide in said biological sample indicates that the subject is unlikely to respond to administration of 5HT₁ agonists.

11. The diagnostic test for determining the efficacy of 5HT₁ agonists as set forth in claim 1, wherein said cranial facial pain syndrome is selected from the group consisting of migraine, temporomandibular joint dysfunction, sinus headache, rhinosinusitis, fibromyalgia, and trigeminal neuralgia.

12. The diagnostic test for determining the efficacy of 5HT₁ agonists as set forth in claim 1, wherein said 5HT₁ agonist is selected from the group consisting of sumatriptan, naratriptan, rizatriptan, zolmitriptan, eletriptan, almotriptan, and frovatriptan.

13. A diagnostic test for determining the efficacy of 5HT₁ agonists in a human patient exhibiting symptoms of a cranial facial pain syndrome, comprising the steps of:
providing at least one antibody corresponding to a biological marker, wherein said biological marker is calcitonin-gene-related peptide;
collecting a sample of saliva from said human patient;
applying said antibody to said saliva sample; and
evaluating said saliva sample for evidence of binding of said antibody with said biological marker present in said saliva sample.

14. The diagnostic test for determining the efficacy of 5HT₁ agonists as set forth in claim 13, wherein the step of providing at least one antibody further comprises providing a test strip containing said at least antibody thereon.

15. The diagnostic test for determining the efficacy of 5HT₁ agonists as set forth in claim 14, wherein said step of providing a test strip further comprises providing said test strip in the form of a biosensor comprising a substrate having an electrical impedance and a coating, said at least one antibody being arranged on said coating.

16. The diagnostic test for determining the efficacy of 5HT₁ agonists as set forth in claim 14, wherein said coating comprises D-poly lysine.

17. The diagnostic test for determining the efficacy of 5HT₁ agonists as set forth in claim 15, wherein said substrate comprises a gold ribbon.

18. The diagnostic test for determining the efficacy of 5HT₁ agonists as set forth in claim 15, wherein said step of evaluating said biosensor further comprises measuring a change in said electrical impedance of said biosensor substrate.

19. The diagnostic test for determining the efficacy of 5HT₁ agonists as set forth in claim 18, further comprising the step of converting said change in said electrical impedance of said biosensor substrate into a transmittable electrical signal.

20. A diagnostic test for determining the efficacy of 5HT₁ agonists in a human patient exhibiting symptoms of a cranial facial pain syndrome, comprising the steps of:
providing at least one antibody corresponding to a biological marker,
collecting a sample of saliva from said human patient;
applying said antibody to said saliva sample; and
evaluating said saliva sample for evidence of binding of said antibody with said biological marker.

21. The diagnostic test for determining the efficacy of 5HT₁ agonists as set forth in claim 20, wherein said biological marker is calcitonin-gene-related peptide;

22. A preemptive prophylaxis method for treatment of a cranial facial pain syndrome in a human patient, comprising the steps of:
collecting a sample of saliva from said human patient;
evaluating said saliva sample for evidence of the presence of calcitonin-gene-related peptide to determine an efficacy of a 5HT₁ agonist in treating said cranial facial pain syndrome in said human patient; and
administering said 5HT₁ agonist to said human patient if said evaluation of said human patient's saliva sample indicates that treatment with said 5HT₁ agonist will be effective.

23. The preemptive prophylaxis method for treatment of a cranial facial pain syndrome as set forth in claim 22, wherein said 5HT₁ agonist is selected from the group consisting of sumatriptan, naratriptan, rizatriptan, zolmitriptan, eletriptan, almotriptan, and frovatriptan.

24. The preemptive prophylaxis method for treatment of a cranial facial pain syndrome as set forth in claim 22, wherein said cranial facial pain syndrome is selected from the group consisting of migraine, temporomandibular joint dysfunction, sinus headache, rhinosinusitis, fibromyalgia, and trigeminal neuralgia.

25. A method for classifying whether a human patient exhibiting symptoms of a cranial facial pain syndrome is likely to respond to administration of a 5HT₁ agonist, said method comprising:
obtaining a sample from said human patient;
determining a diagnostic marker profile by detecting the presence or level of calcitonin-gene-related peptide in said sample.

26. The method for classifying as set forth in claim 25, wherein said step of determining a diagnostic marker profile further comprises:
providing an antibody corresponding to calcitonin-gene-related peptide; and
applying said antibody to said sample.

27. The method for classifying as set forth in claim 25, wherein the presence or level of said at least one diagnostic marker is detected using capillary liquid chromatography-electrospray ionization-mass spectrometry and tandem mass spectrometry.

28. The method for classifying as set forth in claim 25, wherein said step of determining a diagnostic marker profile further comprises:
providing a nucleic acid corresponding to calcitonin-gene-related peptide; and
applying said nucleic acid to said sample.

29. The method for classifying as set forth in claim 28, wherein said nucleic acid is a mirror-imaged aptamer.

30. A biological marker for determining responsiveness of a human patient exhibiting symptoms of a cranial facial pain syndrome to administration of a 5HT₁ agonist, comprising calcitonin-gene-related peptide.

31. The biomarker as set forth in claim 29, wherein said cranial facial pain syndrome is selected from the group consisting of migraine, temporomandibular joint dysfunction, sinus headache, rhinosinusitis, fibromyalgia, and trigeminal neuralgia.

32. A method for determining the efficacy of 5HT₁ agonists for treatment of a cranial facial pain syndrome, said method comprising:
evaluating a biological sample obtained from a subject exhibiting symptoms of said cranial facial pain syndrome for the presence or absence of calcitonin-gene-related peptide, wherein the presence of calcitonin-gene-related peptide in said biological sample indicates that the subject is likely to respond to administration of 5HT₁ agonists.

33. The method for determining the efficacy of 5HT₁ agonists as set forth in claim 32, wherein the absence of calcitonin-gene-related peptide in said biological sample indicates that the subject is unlikely to respond to administration of 5HT₁ agonists.
